(19) United States
(12) Patent Application Publication
(10) Pub. No.: US 2011/0256529 A1
(43) Pub. Date: Oct. 20, 2011

(54) BEADS FOR USE IN REACTIONS FOR THE AMPLIFICATION AND/OR SYNTHESIS OF A POLYNUCLEOTIDE, AND A DEVICE AND A METHOD FOR THE PRODUCTION THEREOF

(76) Inventors: Johanna Kate Daniels, Dinas Powys (GB); Daniel David Palmer, Cardiff (GB); Richard James Calder, Cardiff (GB)

(21) Appl. No.: 12/443,593

(22) PCT Filed: Oct. 2, 2007
(86) PCT No.: PCT/GB07/03734

§ 371 Date: Jul. 8, 2009

(30) Foreign Application Priority Data
Oct. 2, 2006 (GB) 0619442.7

(51) Int. Cl. C12Q 1/68; B01J 19/06; C12M 1/40

(52) U.S. Cl. 435/6.1; 435/289.1; 422/307

(57) ABSTRACT

A method is provided of making meltable beads for use in reactions for the amplification and/or synthesis of a polynucleotide, the method comprising (i) Providing one or more reagents for use in a reaction for the amplification and/or synthesis of a polynucleotide and a substance for promoting the formation of a solid bead; (ii) Providing a reactor device comprising a sample conduit and a first carrier fluid conduit for the flow of immiscible liquids, the sample conduit and first carrier fluid conduit meeting at a junction; (iii) Heating the one or more reagents for use in a reaction for the amplification and/or synthesis of a polynucleotide and the substance for promoting the formation of a solid bead so as to form a liquid comprising the one or more reagents for use in a reaction for the amplification and/or synthesis of a polynucleotide and the substance for promoting the formation of a solid bead in intimate admixture; (iv) Passing the liquid down the sample conduit and a carrier fluid down the first carrier fluid conduit, thus causing the formation of droplets at or downstream of the junction and (v) Causing the formation of solid beads by cooling the droplets. A device for use in such methods

The present invention relates to beads for use in reactions for the amplification and/or synthesis of a polynucleotide, such as (but not exclusively) polymerase chain reaction (PCR) reactions, and a method and a device for making such beads.

Polynucleotide amplification reactions typically use an enzyme (a polymerase) to promote amplification of the polynucleotide sequence in question in conjunction with one or more primers that initiate amplification. In conventional amplification reactions, the reagents are in contact with one another throughout the whole of the reaction process, including prior to the first DNA denaturation step. These reaction conditions can lead to the formation of undesired amplification products. Meltable beads have been used in PCR reactions in order to isolate at least some of the reagents from one another. The techniques which are used to make conventional beads produce beads which are not of a uniform size and so the user does not know the precise volume of reagents contained in the beads which, for certain applications, may cause problems. Furthermore, the shelf-life of conventional beads may be limited. Each of the techniques used to make conventional beads are also only generally suitable for making beads in a small range of sizes.

The present invention is provided to address one or more of these problems.

In accordance with a first aspect of the present invention there is provided a plurality of meltable spherical beads for use in a reaction for the amplification and/or synthesis of a polynucleotide, the beads having a mean diameter of from 50 to 2500 microns, the standard deviation of the diameter of the plurality of beads being no greater than 5% of the mean diameter of the plurality of beads, each of the beads comprising one or more reagents for use in a reaction for the amplification and/or synthesis of a polynucleotide.

These beads have proved to be of particular use in reactions for the amplification of polynucleotides.

The one or more reagents may comprise one or more of an unlabelled oligonucleotide, a labelled oligonucleotide, labelled deoxynucleoside triphosphates, unlabelled deoxynucleoside triphosphates, labelled oxynucleoside triphosphates, unlabelled oxynucleoside triphosphates, enzyme for the amplification and/or synthesis of polynucleotides, magnesium ions, potassium ions, sodium ions, a polynucleotide, a stain or dye and a compound that, in use, produces a buffering effect.

The unlabelled oligonucleotide may be a primer for use in a PCR reaction. The labelled oligonucleotide may be a probe for use in a PCR reaction. Examples of enzymes for the amplification and/or synthesis of polynucleotides are well-known to those skilled in the art, such as Taq® polymerase or reverse transcriptase. The polynucleotide may be the polynucleotide which is to be amplified during the amplification reaction. Such a polynucleotide may be advantageously incorporated into the reagent composition if the reagent composition is to form a positive control sample, for example. The compound that, in use, produces a buffering effect in a reaction for the amplification and/or synthesis of a polynucleotide may be, for example, TRIS-HCl [2-Amino-2-(hydroxymethyl)-1,3-propanediol, hydrochloride].

The stain or dye may be one of those typically used to follow the progress of a polynucleotide amplification reaction. Such dyes or stains may typically be fluorophores. The dyes or stain may be an intercalator (i.e. molecule that becomes bound into the double helix structure). SYBR Green I and ethidium bromide are examples of such intercalators. Alternatively, the dye or stain may be non-intercalating, such as Hoechst 33258.

It is preferred each of the beads comprises an enzyme for the amplification and/or synthesis of polynucleotides, such as Taq polymerase. Each of the beads may comprise an enzyme for the amplification and/or synthesis of polynucleotides and a compound that, in use, produces a buffering effect. Each of the beads may comprise an enzyme for the amplification and/or synthesis of polynucleotides, a compound that, in use, produces a buffering effect and deoxynucleoside triphosphates (labeled or unlabelled).

It is preferred that the beads comprise a substance for promoting the formation of a solid bead. Such a substance typically facilitates the solid, meltable bead to be formed from a liquid droplet, which is a convenient way of producing such a solid bead.

The substance for promoting the formation of a solid bead may comprise one or more of wax or a gelling agent, such as a polysaccharide. The polysaccharide may comprise one or more of Curdlan gum, Carrageenan, β-Glucans, Guar gum, Gellan gum, Gelatin, Locust bean gums, Pectin and Xanthan gum. The polysaccharide may comprise a galactan, such as Locust bean gum, Pectin, Carrageenan, Guar gum and agarose.

The beads may comprise from 0.1 to 10 wt % of the substance for promoting the formation of a solid bead, preferably from 0.3 to 3 wt % of the substance for promoting the formation of a solid bead, more preferably 0.3 to 1.5 wt % of the substance for promoting the formation of a solid bead and further more preferably from 0.4 to 1.2 wt % of the substance for promoting the formation of a solid bead. The percentage compositions have proved to be particularly effective where the substance for promoting the formation of a solid bead is a gelling agent, particularly a polysaccharide and more particularly a galactan, especially agarose.

The mean diameter of the beads may be from 500 to 2500 microns, preferably from 800 to 1500 microns and more preferably from 900 to 1200 microns.

It is preferred that the standard deviation of the diameter of the beads is small. The standard deviation of the diameter of the plurality of beads may be no greater than 3% of the mean diameter and preferably no greater than 2% of the mean diameter.

It is preferred that the melting point of the bead is a predetermined temperature to suit the enzyme being used. Entrapment of the enzyme inhibits the enzyme from assisting in the amplification or synthesis of a polynucleotide, and therefore the melting point of the bead is chosen so that the enzyme is only released when the desired operating temperature is reached. If the enzyme is not entrapped it is free to assist in the amplification and/or synthesis of a polynucleotide, and may therefore generate polynucleotides other than that or those desired. For example, beads entrapping Tag polymerase may be arranged to melt several degrees below the usual operating temperature of the enzyme. In this case, the bead melting may be 90° C. because the usual operating temperature of the enzyme is about 94° C.

It is preferred that the melting point of the beads may be no greater than 100° C. The melting point of the beads may be no less than 30° C. The melting point of the beads may preferably be from 80° C. to 95° C.

It is preferred that the plurality of beads comprises no fewer than 10 beads, preferably no fewer than 30 beads and more preferably no fewer than 100 beads.

The present invention further provided a meltable spherical bead for use in a reaction for the amplification and/or synthesis of a polynucleotide, the meltable spherical bead being selected from a plurality of beads in accordance with the first aspect of the present invention.

In accordance with the second aspect of the present invention, there is provided a plurality of reaction vessels, each reaction vessel containing one or more meltable spherical beads for use in a reaction for the amplification and/or synthesis of a polynucleotide, the beads in the plurality of reaction vessels having a mean diameter of from 50 to 2500 microns, the standard deviation of the diameter of the beads in the plurality of reaction vessels being no greater than 5% of the mean diameter, each of the beads comprising one or more reagents for use in reaction for the amplification and/or synthesis of a polynucleotide.

The meltable spherical beads provided may demonstrate those features as described above in relation to the first aspect of the present invention. For example, beads may comprise a substance for promoting the formation of a solid bead, such as a galactan.

In accordance with a third aspect of the present invention, there is provided a reactor device for the manufacture of meltable beads, the reactor device comprising:

(i) a sample conduit for the flow of a first liquid which, on cooling, may form a meltable bead,

(ii) a first carrier fluid conduit for the flow of a carrier liquid, the sample conduit and the first carrier fluid conduit meeting at a junction

(iii) a segmented flow conduit which leads away from the junction, the segmented flow conduit having a proximal portion associated with the junction and a distal portion downstream of the junction, wherein at or adjacent to the junction, the reactor device is provided with a flow constriction or discontinuity that, in use, causes the formation of segments of the first liquid in the carrier liquid,

(iv) a heater for heating the sample conduit and the first carrier fluid conduit, the junction and optionally the proximal portion of the segmented flow conduit and

(v) a cooler for cooling the distal portion of the segmented flow conduit so to promote the solidification of the segments of the first liquid.

The device of the present invention provides an effective device for making the beads of the first aspect of the present invention by cooling the segments of the first liquid to form solid beads.

The reactor device may further be provided with a second carrier fluid conduit meeting the sample conduit at the junction.

The reactor device is preferably a microfluidic device. The term “microfluidic” is generally well-understood by those skilled in the art. The conduits in such microfluidic devices typically have widths of no greater than 2 mm, and preferably from 0.5 to 2 mm. The depths of the conduits are typically less than 2 mm and preferably from 0.5 mm to 2 mm. The flow rates of the fluids will depend, inter alia, on the cross-sectional area of the conduits, and the preferred values given here relate to conduits having depths of from 1 mm and 2 mm and widths of from 1 mm to 2 mm. The flow rate, for example, of the first liquid through the sample conduit may advantageously be from about 0.02 to 5 ml/hour, more preferably be from about 0.1 to 2 ml/hour. The flow rate of the carrier fluid may be from about 0.2 to 60 ml/hour, preferably from about 0.2 to 15m1/hour and more preferably from about 1 to 3 ml/hour.

One or more (and preferably all) of the sample conduit, first and second carrier fluid conduits, and the segmented flow conduit may be provided in the surface of a substrate, such as a block. This provides a convenient reactor device, and may typically be formed by machining conduits into a substrate. The substrate may comprise a low energy material, such as polytetrafluoroethylene (PTFE), an example of which is Teflon. Alternatively, conduits may be formed in a substrate by machining a substrate of a relatively high surface energy and coating the machined substrate with a low energy material to provide passages having surfaces of a low energy.

The reactor device is preferably provided with a device outlet for the egress of solid beads.

The reactor device may be provided with one or more flushing conduits for providing a flushing liquid to the device outlet to inhibit blocking of the outlet with solid beads.

It is preferred that the one or more flushing conduits meet the segmented flow conduit upstream of the device outlet. The one or more flushing conduits may meet the segmented flow conduit in proximity to the device outlet. For example, the one or more flushing conduits may meet the segmented flow conduit at a distance of less than 20 times the width of the segmented flow conduit upstream of the device outlet (where the width of the segmented flow conduit is that of the segmented flow conduit where it meets the one or more flushing conduits). It is preferred that the reactor device is provided with two flushing conduits which meet the segmented flow conduit at a flushing junction.

It is preferred that the one or more flushing conduits is in fluid communication with a flushing inlet for introducing fluid into the one or more flushing conduits. It is preferred that there is only one flushing inlet.

The flushing liquid may be the same as the carrier fluid used in the formation of beads.

The device may be provided with a heating plate for heating the sample conduit and the first carrier fluid conduit, the junction, the second carrier fluid conduit (if present) and optionally the proximal portion of the segmented flow conduit.

The device may be provided with a cooling plate for cooling the distal portion of the segmented flow conduit. The cooling plate is a convenient way of forming a good thermal contact with other parts of the device, especially if the conduits are formed in a substrate or block.

It is preferred that the device is provided with a heating plate and a cooling plate. If this is the case, then it is preferred that there is an insulating gap provided between the heating plate and the cooling plate. The insulating gap may comprise an insulating gas, such as air. The insulating gap is preferably of from 0.1 to 10 mm. The space between the cooling plate and the heating plate may be from 0.1 to 10 mm.

The length of the segmented flow conduit in thermal communication with the cooler may be from 2 to 5 times the length of the segmented flow conduit in thermal communication with the heater.

The segmented flow conduit may be provided with a step downstream of the junction. The step may assist in the formation of spherical beads.

The step may be located at a distance of from 0.5 a to 5 a downstream of the point at which the segmented flow conduit meets the junction, “a” being the depth of the segmented flow conduit immediately upstream of the step. The step may preferably be located at a distance of from 0.5 a to 2.0 a (and more preferably from 0.5 a to 1.5 a) downstream of the point at which the segmented flow conduit meets the junction, “a” being the depth of the segmented flow conduit immediately upstream of the step. These device geometries have proved to be effective in producing spherical beads.

The segmented flow conduit may be provided with an enlargement in cross-section downstream of the point at which the segmented flow conduit meets the junction. Such an enlargement in cross-section may produce a step at the enlargement.

The enlargement in cross-section may be located at a distance of from 0.25 d to 2.5 d downstream of the point at which the segmented flow conduit meets the junction, “d” being the depth of the segmented flow conduit immediately downstream of the enlargement. The enlargement in cross-section is preferably located at a distance of from 0.25 d to d (and more preferably 0.25 d to 0.75 d) downstream of the point at which the segmented flow conduit meets the junction.

The cross-sectional area of the segmented flow conduit immediately downstream of the enlargement may be from 1.5 to 6 times (and preferably from 2 to 5 times) the cross-sectional area of the segmented flow conduit immediately upstream of the enlargement.

In accordance with a fourth aspect of the present invention, there is provided a method of making meltable beads for use in reactions for the amplification and/or synthesis of a polynucleotide, the method comprising

(i) Providing one or more reagents for use in a reaction for the amplification and/or synthesis of a polynucleotide and a substance for promoting the formation of a solid bead;

(ii) Providing a reactor device comprising a sample conduit and a first carrier fluid conduit for the flow of immiscible liquids, the sample conduit and the first carrier fluid conduit meeting at a junction;

(iii) Heating the one or more reagents for use in a reaction for the amplification and/or synthesis of a polynucleotide and the substance for promoting the formation of a solid bead so as to form a liquid comprising the one or more reagents for use in a reaction for the amplification and/or synthesis of a polynucleotide and the substance for promoting the formation of a solid bead in intimate admixture;

(iv) Passing the liquid down the sample conduit and a carrier fluid down the first carrier fluid conduit, thus causing the formation of droplets at or downstream of the junction; and

(v) Causing the formation of solid beads by cooling the droplets.

This method provides an effective method of forming beads, especially spherical beads. This method has further proved effective in producing beads that have a low polydispersity i.e. small variation in size and is effective at producing beads over a wide range of desired sizes.

The reactor device is preferably the reactor device in accordance with the third aspect of the present invention, and therefore may incorporate those features described above with respect to the reactor device of the third aspect of the present invention.

This method may be used to form the beads of the first aspect of the present invention. Therefore, the statements made in relation to bead composition with reference to the beads of the first aspect of the present invention also pertain to the method of the fourth aspect of the present invention.

The one or more reagents for use in a reaction for the amplification and/or synthesis of a polynucleotide are preferably those described above with reference to the beads of the first aspect of the present invention. For example, the one or more reagents may comprise one or more of an unlabelled oligonucleotide, a labelled oligonucleotide, labelled deoxynucleoside triphosphates, unlabelled deoxynucleoside triphosphates, labelled oxynucleoside triphosphates, unlabelled oxynucleoside triphosphates, enzyme for the amplification and/or synthesis of polynucleotides, magnesium ions, potassium ions, sodium ions, a polynucleotide, a stain or dye and a compound that, in use, produces a buffering effect.

The unlabelled oligonucleotide may be a primer for use in a PCR reaction. The labelled oligonucleotide may be a probe for use in a PCR reaction. Examples of enzymes for the amplification and/or synthesis of polynucleotides are well-known to those skilled in the art, such as Taq® polymerase or reverse transcriptase. The polynucleotide may be the polynucleotide which is to be amplified during the amplification reaction. Such a polynucleotide may be advantageously incorporated into the reagent composition if the reagent composition is to form a positive control sample, for example. The compound that, in use, produces a buffering effect in a reaction for the amplification and/or synthesis of a polynucleotide may be, for example, TRIS-HCl [2-Amino-2-(hydroxymethyl)-1,3-propanediol, hydrochloride].

The stain or dye may be one of those typically used to follow the progress of a polynucleotide amplification reaction. Such dyes or stains may typically be fluorophores. The dyes or stains may be an intercalator (i.e. molecule that becomes bound into the double helix structure). SYBR Green I and ethidium bromide are examples of such intercalators. Alternatively, the dye or stain may be non-intercalating, such as Hoechst 33258.

The liquid formed in step (iii) is preferably aqueous. The carrier fluid is therefore preferably a fluid immiscible with an aqueous mixture, solution or suspension.

Furthermore, the substance for promoting the formation of a solid bead may comprise one or more of wax or a gelling agent, such as a polysaccharide. The polysaccharide may comprise one or more of Curdlan gum, Carrageenan, (3-Glucans, Guar gum, Gellan gum, Gelatin, Locust bean gums, Pectin and Xanthan gum. The polysaccharide may comprise a galactan, such as Locust bean gum, Pectin, Carrageenan, Guar gum and agarose.

The liquid formed in step (iii) may comprise from 0.1 to 10 wt % of the substance for promoting the formation of a solid bead, more preferably from 0.3 to 3 wt % of the substance for promoting the formation of a solid bead, more preferably 0.3 to 1.5 wt % of the substance for promoting the formation of a solid bead and most preferably from 0.4 to 1.2 wt % of the substance for promoting the formation of a solid bead. These ranges are especially beneficial if the substance for promoting the formation of a solid bead is a gelling agent, such as a galactan.

It is preferred that the melting point of the bead is a predetermined temperature to suit the enzyme being used. Entrapment of the enzyme inhibits the enzyme from assisting in the amplification or synthesis of a polynucleotide, and therefore the melting point of the bead is chosen so that the enzyme is only released when the desired operating temperature is reached. If the enzyme is not entrapped it is free to assist in the amplification and/or synthesis of a polynucleotide, and may therefore generate polynucleotides other than that or those desired

It is preferred that the device is provided with a segmented flow conduit which leads away from the junction, the liquid beads being cooled in the segmented flow conduit. It is preferred that the segmented flow conduit has a proximal portion associated with the junction and a distal portion downstream of the junction.

In accordance with a fifth aspect of the invention, there is provided an aqueous composition for the formation of meltable beads for use in a reaction for the amplification and/or synthesis of a polynucleotide, the composition comprising a substance for promoting the formation of a solid bead and one or more reagents for use in a reaction for the amplification and/or synthesis of a polynucleotide, the composition comprising 0.1 to 10 wt % of the substance for promoting the formation of a solid bead.

The composition preferably comprises from 0.3 to 3 wt % of the substance for promoting the formation of a solid bead, more preferably 0.3 to 1.5 wt % of the substance for promoting the formation of a solid bead and further more preferably from 0.4 to 1.2 wt % of the substance for promoting the formation of a solid bead. These ranges are especially beneficial if the substance for promoting the formation of a solid bead is a gelling agent, such as a polysaccharide.

The device may be a microfluidic device.

The device may comprise a second carrier fluid conduit which meets the sample conduit, preferably at the junction.

The composition in accordance with the fifth aspect of the present invention may be used to make the beads of the first aspect of the present invention, for example, using the method of the fourth aspect of the present invention. The composition of the fifth aspect of the present invention may be the liquid used in the fourth aspect of the present invention. The statements made in relation to the composition of the beads of the first aspect of the present invention may also apply to the mixture of the fifth aspect of the present invention.

For example, the one or more reagents for use in a reaction for the amplification and/or synthesis of a polynucleotide may comprise one or more of the following: an unlabelled oligonucleotide, a labelled oligonucleotide, labelled deoxynucleoside triphosphates, unlabelled deoxynucleoside triphosphates, labelled oxynucleoside triphosphates, unlabelled oxynucleoside triphosphates, enzyme for the amplification and/or synthesis of polynucleotides, magnesium ions, potassium ions, sodium ions, a polynucleotide, a stain or dye and a compound that, in use, produces a buffering effect.

The present invention will now be described by way of example only with reference to the following Figures, of which:

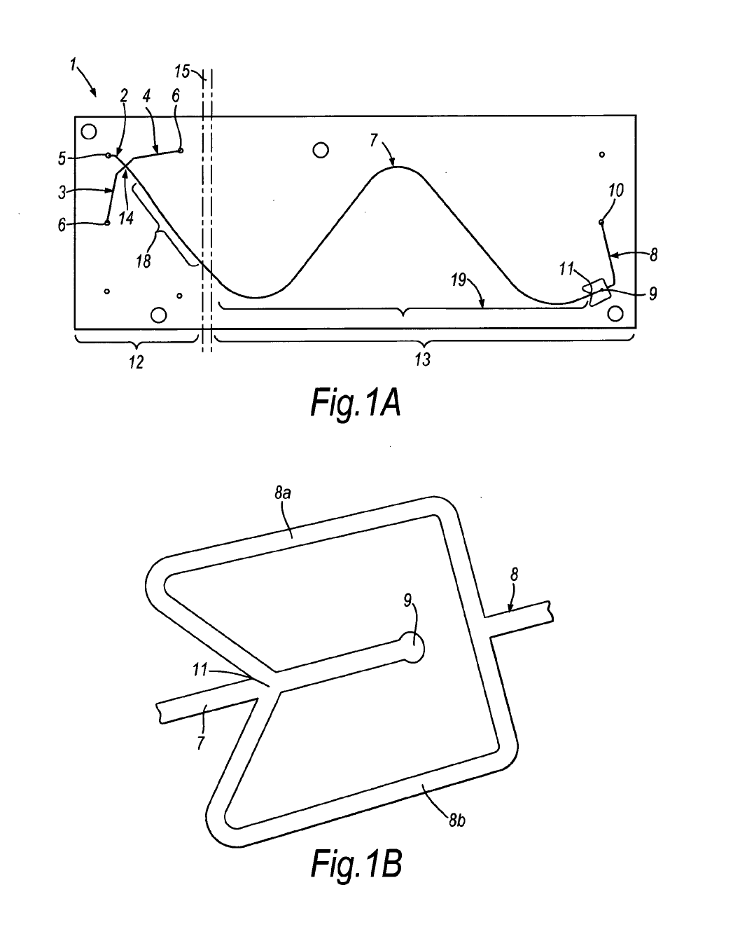

FIG. 1, which is a plan view of a reactor device in accordance with the present invention;

FIG. 1B is a plan view of the portion of the device of FIG. 1 around the device outlet showing the flushing arrangement;

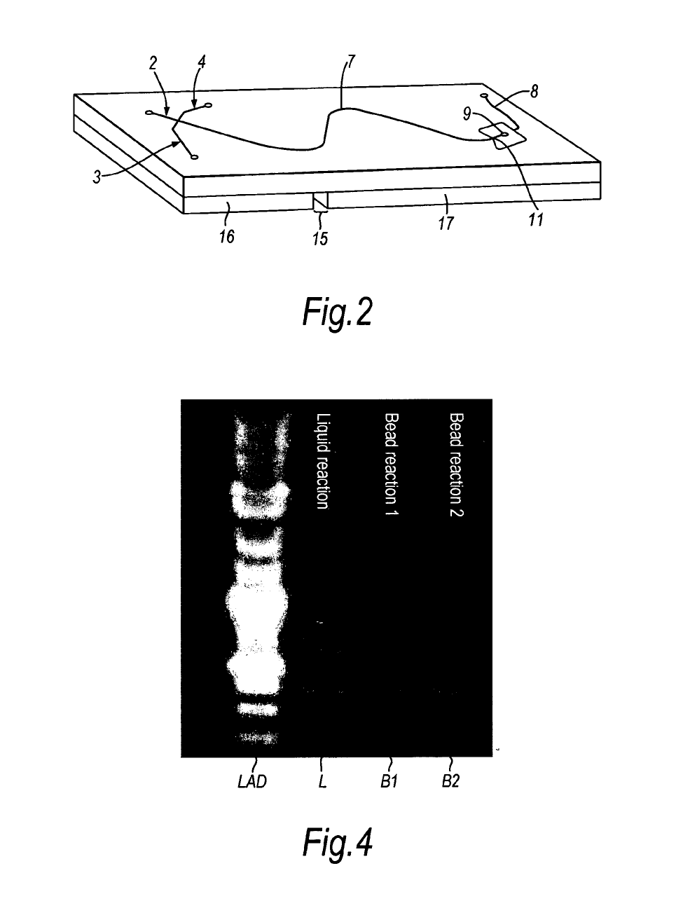

FIG. 2, which is a schematic perspective view of the reactor device of FIG. 1;

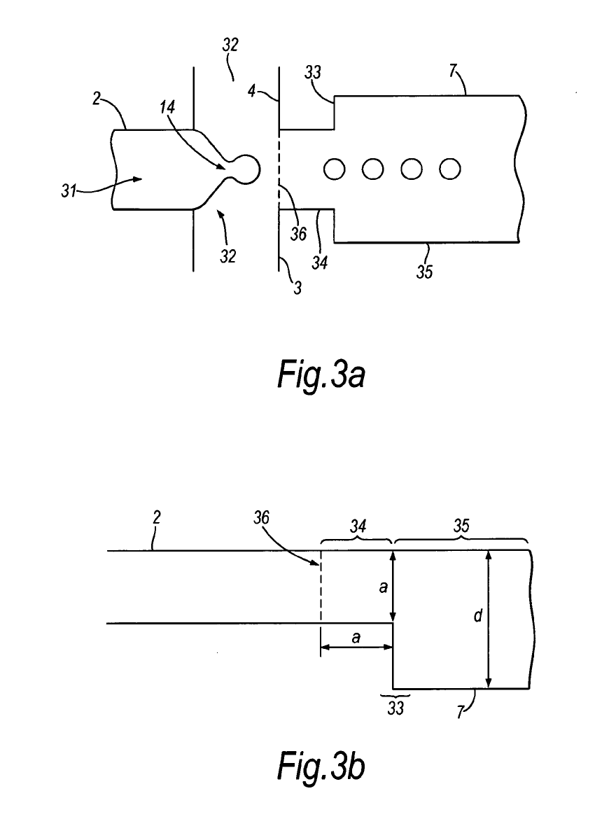

FIG. 3a is a plan view of the region of the reactor device around the junction;

FIG. 3b is a cross-sectional view of the reactor device around the junction; and

FIG. 4 shows electrophoresis images of gel plates of PCR products demonstrating the effect of entrapping one or more reagents in a solid bead compared to one or more reagents stored as a liquid.

FIG. 1 shows a reactor device in accordance with the present invention, the reactor device shown generally by reference numeral 1. The reactor device 1 comprises a sample conduit 2 for the flow of a first liquid which, on cooling, may form a meltable bead and a first carrier fluid conduit 4 for the flow of a carrier liquid, the sample conduit 2 and first carrier fluid conduit 4 meeting at a junction 14. A segmented flow conduit 7 leads away from the junction, the segmented flow conduit 7 having a proximal portion 18 associated with the junction 14 and a distal portion 19 downstream of the junction. The reactor device is further provided with a second carrier fluid conduit 3 for the flow of a carrier liquid. The second carrier fluid conduit 3 meets the sample conduit 2 at the junction 14. At or adjacent to the junction, the reactor device is provided with a flow constriction or discontinuity that, in use, causes the formation of segments of first liquid in the carrier fluid. In the present case, the junction itself causes a flow constriction; referring to FIGS. 3a and 3b, the cross-section of the portion 34 of the segmented flow conduit 7 immediately downstream of the junction 14 is the same as each of the sample conduit 2 and the first and second carrier fluid conduits. This causes a constriction in flow, which causes the formation of segments of the first liquid in the carrier fluid. The reactor device 1 is further provided with a heater (not shown) for heating the sample conduit and the first carrier fluid conduit, the junction and the proximal portion 18 of the segmented flow conduit. The reactor device is further provided with a cooler (not shown) for cooling the distal portion 19 of the segmented flow conduit so to promote the solidification of the segments of the first liquid.

Referring to FIG. 2, the heater (not shown) is in thermal communication with a heating plate 16 which is abutted against, and fixed to, the underside of the polytetrafluoroethylene (PTFE) substrate in which the sample conduit 2, the first and second carrier fluid conduits, and the segmented flow conduit 7 have been formed. Similarly, the cooler (in this case, a cold water pumping system) is in thermal communication with a cooling plate 17 which is abutted against, and fixed to, the underside of the polytetrafluoroethylene (PTFE) substrate. The heating plate 16 and cooling plate 17 are separated by a small insulating gap 15 (in this case, provided by 5 mm of air). The insulating gap 15 reduces thermal communication between the heating plate 16 and cooling plate 17. This separation further ensures that junction region 14 and region 18 of the segmented flow conduit 7 are not significantly cooled by the cooling plate 17, and, likewise, the region 19 of the segmented flow conduit 7 is not significantly heated by the heating plate 16.

The geometry of the junction 14 is shown in greater detail in FIGS. 3a and 3b. The first and second carrier fluid conduits meet sample conduit 2 at a junction 14 and the segmented flow conduit 7 leads away from the junction 14. The segmented flow conduit 7 is provided with an enlargement 33 in cross-section downstream of the junction 14. The depth of the portion 34 of the segmented flow conduit 7 upstream of the enlargement 33 is about 0.7 times the depth of the portion 35 of the segmented flow conduit 7 immediately downstream of the enlargement 33. Furthermore, the width of the portion 34 of the segmented flow conduit 7 upstream of the enlargement 33 is 1.25 mm, about 0.7 times the width of the portion 35 of the segmented flow conduit 7 immediately downstream of the enlargement 33 (1.7 mm). The distance from the point 36 at which the segmented flow conduit 7 joins the junction 14 to the enlargement 33 is about the same as the depth of portion 34. Furthermore, the depth of the segmented flow conduit 7 immediately downstream of the enlargement 33 is 1.7 mm, about 1.4 times the depth of the segmented flow conduit 7 immediately upstream of the enlargement 33 (1.25 mm). This enlargement 33 and its position relative to the junction 14 have proved to be beneficial in assisting the formation of spherical beads. The sample conduit 2 and the carrier fluid conduits 3, 4 each have a depth and width of 1.25 mm. The size of the conduits helps define the size of the beads that are produced, and so conduits of different widths and depths may be used to produce beads of different sizes.

### EXAMPLE 1

An example of a method of producing meltable beads in accordance with the present invention will now be described with reference to FIGS. 1, 2, 3a and 3b.

An aqueous solution of Taq® polymerase and agarose (Sigma-Aldrich, Gillingham, Dorset, UK) was prepared as follows. A known amount of agarose was added to a known amount of water, 11 mg of agarose being added per 1000 μl of water. Dissolution of the agarose was encouraged by heating the water and agarose in a Falcon tube in a microwave oven. The Falcon tube was then put into a water bath at 70° C. 104 μl of Taq® polymerase (Helena Biosciences, Gateshead, UK) was measured into an eppendorf tube which was put into the water bath at 70° C.

The eppendorf tube and the Falcon tube were removed from the water bath. 948 μl of agarose solution were pipetted into the eppendorf tube containing the Taq® polymerase and the contents of the eppendorf tube were then vortex-mixed for 2-4 seconds at 2700 rpm. The eppendorf tube was then inserted into a Spectrafuge centrifuge and the contents of the eppendorf tube centrifuged for about 5 to 10 seconds.

The eppendorf tube is then returned to the water bath at 70° C. The eppendorf tube should be out of the water bath for a duration of less than 30 seconds.

A pre-heated 1 ml syringe is then loaded with the Taq®/agarose solution, and the syringe is then inserted into a syringe heater (not shown) for maintaining the syringe and its contents at about 65° C. The syringe is connected to the inlet 5. Three 10 ml syringes are each loaded with mineral oil (Sigma-Aldrich, Gillingham, Dorset, UK). Each of these three syringes is connected to one of the carrier fluid inlets 6 and the flushing inlet 10. Liquid is released from each of the syringes by automated syringe drivers. Carrier fluid 32 is urged into carrier fluid inlets 6 into the first 3 and second 4 carrier fluid conduits at a flow rate of 10 ml/hour. The aqueous solution 31 of agarose and Taq® polymerase is urged into the sample conduit 2 at a flow rate of 1.3 ml/hour. Flushing fluid is urged into the flushing conduit 8 at a flow rate of 15 ml/hour.

Referring to FIGS. 3a and 3b, when the carrier fluid 32 and aqueous solution 31 meet at the junction 14, a necked-droplet shape of aqueous solution is seen to form. As the necked-droplet shape moves downstream, the neck is seen to narrow and eventually break giving isolated droplets. The isolated droplets are seen to form downstream of the junction. The enlargement 33 appears to promote the formation of spherical droplets.

Referring to FIG. 1, the region 18 of the segmented flow conduit 7 is heated to promote the formation of spherical droplets. Region 19 of the segmented flow conduit 7 is cooled by passing ice-cooled water over the underside of metal plate 17.

Liquid droplets pass into cooled region 19 and cool, forming solid beads. The solid beads are carried by the carrier fluid 32 through device outlet 9 and out of the device 1.

The device 1 is provided with a flushing arrangement for inhibiting the blockage of the device outlet 9 by solid beads. FIG. 1B shows a plan view of the region of the device 1 around the device outlet 9. Referring to FIG. 1 and FIG. 1B, solid beads pass along segmented flow conduit 7 towards device outlet 9. Flushing fluid (in this case, the same as the carrier fluid 32) is passed from a syringe (not shown) to conduit 8 via flushing inlet 10. Conduit 8 supplies flushing conduits 8a, 8b with flushing fluid. Flushing conduits 8a, 8b meet segmented flow conduit 7 at a flushing junction 11 in proximity to device outlet 9. The introduction of flushing fluid at a relatively high flow rate to the segmented flow conduit 7 just upstream of the device outlet 9 urges solid beads out of the device outlet 9 and inhibits blocking of the device outlet 9.

The solid beads were then stored as appropriate.

### EXAMPLE 2

The method of Example 1 was repeated to manufacture beads, each comprising 0.1 unit of Taq® polymerase. One bead was then placed in each well of a PCR “strip” and 5 μl of a PCR “master mix” added to each well. The master mix included a Qiagen buffer, MgCl₂ solution, a dNTP mix, water, PBR322 plasmid and Sal1 and BamH1 primers. This procedure was replicated for a total of five wells. A positive control sample was run simultaneously with the samples containing the beads. The positive control was made by adding Taq® polymerase solution (as opposed to the bead containing Taq® polymerase) to the PCR master mix. The wells were sealed with caps, placed in an MJ Research thermal cycler and a thermal cycling sequence performed. The thermal cycling sequence used was:

(i) Initial denaturation −94° C. for 5 mins.

(ii) 30 cycles of 3-step cycling, each cycle comprising the steps of:—denaturation —94° C.-30 secs
annealing—55° C.-30 secs
extension—72° C.-1 min

(iii) Final extension—72° C.-10 mins.

Eletrophoresis of the PCR products was then performed on agarose gels in an electrophoresis tank with a Thermo EC power supply. Products were visualized on the gel with an ultra-violet trans illuminator.

Electrophoresis indicated that each of the five samples extracted from the PCR wells showed the same electrophoresis band as the positive control, indicating that effective amplification had taken place.

This example illustrates that very small reaction volumes may be used using the beads of the present invention.

### EXAMPLE 3

Meltable beads were manufactured as described with reference to Examples 1 and 2, and PCR reactions were performed as described above, in parallel with a positive control sample as described above. Referring to FIG. 4, “L” denotes the positive control sample, “LAD” denotes a 2-log DNA ladder reference sample, “B1” and “B2” denote two samples produced using the meltable beads in accordance with the present invention. It can be clearly seen that the beads produce PCR products that give only one electrophoresis band, indicating that only one amplification product is generated. The positive control sample shows at least three electrophoresis bands, indicating that at least three amplification products are produced by the PCR reaction. The increased amplification specificity produced using the beads of the present invention is believed to be due to the Taq® polymerase being separated from the reagents in the PCR mastermix until the Tag is released when the bead melts at above 90° C.

### EXAMPLE 4

Examples 1 to 3 demonstrate how a polymerase may be entrapped in a bead. It is also possible to incorporate one or more primers in the beads of the present invention.

150 μl of a primer solution (stock concentration=10 picomoles/μl) of Sal1 and 150 μl of a primer solution (stock concentration=10 picomoles/μl) of BAMH1 were added to 300 μl of a 2% by weight solution of molten agarose solution (held at 70° C.). This generated a solution containing 1% by weight of agarose and 1500 pmol of each primer.

Beads comprising the primers were manufactured using the device described with reference to FIGS. 1, 1B, 2, 3A and 3B. The flow rate of the agarose/primer solution fed into sample conduit 2 was 1.86 ml/hour. The flow rate of the carrier fluid fed into first and second carrier fluid conduits 3, 4 was 20 ml/hour. The flow rate of the flushing fluid fed into flushing conduits 8a, 8b was 15 ml/hour.

The solid beads were collected and the size of the beads was measured using a Motic microscope in combination with Motic Images Plus 2.0 software. The spherical beads produced were found to have a diameter of 1230±30 μm. It can be calculated that the each bead contains 3.08 pmole of each of the primers.

The primer-containing beads were used in PCR reactions similar to those described above in relation to Example 2. The desired amplification product was produced as confirmed by electrophoresis imaging.

### EXAMPLE 5

The method of examples 1 and 2 was modified to incorporate Qiagen SYBR Green PCR Master Mix into beads. The beads formed had a nominal diameter of 1000 mm, and therefore contained about 526 nl of Master Mix. Thermal cycling as described above in Example 2 was performed on samples comprising 22 Master Mix-containing beads and a pBR3222 template. The same thermal cycling regime was performed on a positive control sample containing a solution of the Master Mix. Electrophoresis showed that the beads produced equivalent amplification to the positive control sample.

### EXAMPLE 6

Beads comprising Taq® polymerase were formed as described in Example 2. Three beads (totaling 0.5 units of Taq® polymerase) were added to a PCR Master Mix comprising all reagents required for the amplification of the human beta-actin gene, including a sample of cDNA from a donor and primers for amplification of the beta-actin gene. After thermal cycling, electrophoresis indicated that the PCR reaction had been successful. This indicates that the beads of the present invention may be used to amplify human genes from cDNA.

### EXAMPLE 7

Beads comprising Taq® polymerase were formed as described in Example 2. A One-Step reverse transcriptase PCR reaction was performed as described hereafter: a polyT primer was annealed to template RNA by heating to 70° C. and crash-cooling to 0° C. 5 μl of the primed RNA was then added to a RT-PCR master mix comprising beads in accordance with the present invention, the beads incorporating Taq® polymerase. The reaction mixture was maintained at 25° C. for 5 minutes before being heated to 42° C. The reaction mixture was maintained at this temperature for 60 minutes, facilitating the reverse transcription of cDNA from the RNA template. The sample was then heated to 70° C., and maintained at this temperature for 15 minutes. At this temperature, the RNA-cDNA duplexes melted and duplexes of cDNA may be formed. The cDNA is amplified by performing 30 cycles of 3-step cycling, each cycle comprising the steps of denaturation at 94° C. for 30 seconds, annealing at 60° C. for 30 seconds and then extension at 72° C. for 1 min. A final extension step of 5 mins. at 72° C. is performed. A positive control gene was simultaneously amplified using positive control primers. Electrophoresis confirmed that the samples comprising the beads in accordance with the present invention produced the desired amplification product. Some higher molecular weight DNA bands were observed possibly due to mispriming.

All of the conduits in the device of FIGS. 1, 2, 3a and 3b were manufactured by machining conduits from a block of low surface energy polymer, in this case polytetrafluoroethlyene.

It will be readily apparent to those skilled in the art that the method of making the beads of the present invention may be performed using devices other than that described above.

1. A method of making meltable beads for use in reactions for the amplification and/or synthesis of a polynucleotide, the method comprising
(i) Providing one or more reagents for use in a reaction for the amplification and/or synthesis of a polynucleotide and a substance for promoting the formation of a solid bead;
(ii) Providing a reactor device comprising a sample conduit and a first carrier fluid conduit for the flow of immiscible liquids, the sample conduit and first carrier fluid conduit meeting at a junction;
(iii) Heating the one or more reagents for use in a reaction for the amplification and/or synthesis of a polynucleotide and the substance for promoting the formation of a solid bead so as to form a liquid comprising the one or more reagents for use in a reaction for the amplification and/or synthesis of a polynucleotide and the substance for promoting the formation of a solid bead in intimate admixture;
(iv) Passing the liquid down the sample conduit and a carrier fluid down the first carrier fluid conduit, thus causing the formation of droplets at or downstream of the junction and
(v) Causing the formation of solid beads by cooling the droplets.

2. A method according to claim 1 wherein the device is provided with a segmented flow conduit which leads away from the junction, the liquid beads being cooled in the segmented flow conduit.

3. A method according to claim 1 wherein the device is a microfluidic device.

4. A method according to claim 1 wherein the device comprises a second carrier fluid conduit which meets the sample conduit.

5. (canceled)

6. A method according to claim 1 wherein the reactor device is a microfluidic device comprising:
(a) a sample conduit for the flow of a first liquid which, on cooling, may form a meltable bead,
(b) a first carrier fluid conduit for the flow of a carrier liquid, the sample conduit and first carrier fluid conduit meeting at a junction
(c) a segmented flow conduit which leads away from the junction, the segmented flow conduit having a proximal portion associated with the junction and a distal portion downstream of the junction, wherein at or adjacent to the junction, the reactor device is provided with a flow constriction or discontinuity that, in use, causes the formation of segments of first liquid in the carrier fluid,
(d) a heater for heating the sample conduit and the first carrier fluid conduit, the junction and optionally the proximal portion of the segmented flow conduit and
(e) a cooler for cooling the distal portion of the segmented flow conduit so to promote the solidification of the segments of the first liquid.

7. A method according to claim 1, wherein the one or more reagents for use in a reaction for the amplification and/or synthesis of a polynucleotide comprises one or more of an unlabelled oligonucleotide, a labelled oligonucleotide, labelled deoxynucleoside triphosphates, unlabelled deoxynucleoside triphosphates, labelled oxynucleoside triphosphates, unlabelled oxynucleoside triphosphates, enzyme for the amplification and/or synthesis of polynucleotides, magnesium ions, potassium ions, sodium ions, a polynucleotide, a stain or dye and a compound that, in use, produces a buffering effect.

8. A method according to claim 1 wherein the liquid formed in step (iii) is aqueous, and the carrier fluid is a fluid immiscible with an aqueous mixture, solution or suspension.

9. (canceled)

10. (canceled)

11. A method according to claim 1, wherein the liquid formed in step (iii) comprises from 0.1 to 10 wt % of the substance for promoting the formation of a solid bead.

12. A method according to any one preceding claim 1, wherein the method forms a plurality of meltable spherical beads for use in a reaction for the amplification and/or synthesis of a polynucleotide, the beads having a mean diameter of from 50 to 2500 microns, the standard deviation of the diameter of the plurality of beads being no greater than 5% of the mean diameter of the plurality of beads, each of the beads comprising one or more reagents for use in reaction for the amplification and/or synthesis of a polynucleotide.

13. A microfluidic reactor device for the manufacture of meltable beads, the reactor device comprising:
(i) a sample conduit for the flow of a first liquid which, on cooling, may form a meltable bead,
(ii) a first carrier fluid conduit for the flow of a carrier liquid, the sample conduit and the first carrier fluid conduit meeting at a junction
(iii) a segmented flow conduit which leads away from the junction, the segmented flow conduit having a proximal portion associated with the junction and a distal portion downstream of the junction, wherein at or adjacent to the junction, the reactor device is provided with a flow constriction or discontinuity that, in use, causes the formation of segments of the first liquid in the carrier fluid,
(iv) a heater for heating the sample conduit and the first carrier fluid conduit, the junction and optionally the proximal portion of the segmented flow conduit and
(v) a cooler for cooling the distal portion of the segmented flow conduit so to promote the solidification of the segments of the first liquid.

14. A reactor device according to claim 13 provided with one or more flushing conduits for providing a flushing liquid to the device outlet to inhibit blocking of the outlet with solid beads.

15. A reactor device according to claim 14 wherein the one or more flushing conduits meet the segmented flow conduit upstream of the device outlet.

16-21. (canceled)

22. A reactor device according to claim 13 wherein the device is provided with a heating plate for heating the sample conduit and the first carrier fluid conduit, the junction and the proximal portion of the segmented flow conduit, and a cooling plate for cooling the distal portion of the segmented flow conduit, wherein there is an insulating gap provided between the heating plate and the cooling plate.

23. A reactor device according to claim 13 wherein the length of the segmented flow conduit in thermal communication with the cooler is from 2 to 5 times the length of the segmented flow conduit in thermal communication with the heater.

24. A reactor device according to claim 13 wherein the segmented flow conduit is provided with a step downstream of the junction, wherein the step is located at a distance of from 1 a to 5 a downstream of the point at which the segmented flow conduit meets the junction, “a” being the depth of the segmented flow conduit immediately upstream of the step.

25. A plurality of meltable spherical beads for use in a reaction for the amplification and/or synthesis of a polynucleotide, the beads having a mean diameter of from 50 to 2500 microns, the standard deviation of the diameter of the plurality of beads being no greater than 5% of the mean diameter of the plurality of beads, each of the beads comprising one or more reagents for use in reaction for the amplification and/or synthesis of a polynucleotide

26. A plurality of meltable spherical beads according to claim 25 wherein the one or more reagents for use in a reaction for the amplification and/or synthesis of a polynucleotide comprises one or more of the following: an unlabelled oligonucleotide, a labelled oligonucleotide, labelled deoxynucleoside triphosphates, unlabelled deoxynucleoside triphosphates, labelled oxynucleoside triphosphates, unlabelled oxynucleoside triphosphates, enzyme for the amplification and/or synthesis of polynucleotides, magnesium ions, potassium ions, sodium ions, a polynucleotide, a dye or stain, and a compound that, in use, produces a buffering effect.

27. (canceled)

28. A plurality of meltable spherical beads according to claim 25 wherein the beads comprise from 0.1 to 10 wt % of-the a substance for promoting the formation of a solid bead.

29. (canceled)

30. (canceled)

31. A plurality of reaction vessels, each reaction vessel containing one or more meltable spherical beads for use in a reaction for the amplification and/or synthesis of a polynucleotide, the beads in the plurality of reaction vessels having a mean diameter of from 50 to 2500 microns, the standard deviation of the diameter of the beads in the plurality of reaction vessels being no greater than 5% of the mean diameter, each of the beads comprising one or more reagents for use in reaction for the amplification and/or synthesis of a polynucleotide.

32. An aqueous composition for the formation of meltable beads for use in a reaction for the amplification and/or synthesis of a polynucleotide, the composition comprising a substance for promoting the formation of a solid bead and one or more reagents for use in a reaction for the amplification and/or synthesis of a polynucleotide, the composition comprising 0.1 to 10 wt % of the substance for promoting the formation of a solid bead.

33. A plurality of meltable spherical beads for use in a reaction for the amplification and/or synthesis of a polynucleotide, the plurality of beads being produced using a method comprising:
(i)Providing one or more reagents for use in a reaction for the amplification and/or synthesis of a polynucleotide and a substance for promoting the formation of a solid bead;
(ii) Providing a reactor device comprising a sample conduit and a first carrier fluid conduit for the flow of immiscible liquids, the sample conduit and first carrier fluid conduit meeting at a junction;
(iii) Heating the one or more reagents for use in a reaction for the amplification and/or synthesis of a polynucleotide and the substance for promoting the formation of a solid bead so as to form a liquid comprising the one or more reagents for use in a reaction for the amplification and/or synthesis of a polynucleotide and the substance for promoting the formation of a solid bead in intimate admixture;
(iv) Passing the liquid down the sample conduit and a carrier fluid down the first carrier fluid conduit, thus causing the formation of droplets at or downstream of the junction; and
(v) Causing the formation of solid beads by cooling the droplets.
